(19) United States
(12) Patent Application Publication
(10) Pub. No.: US 2005/0014716 A1
(43) Pub. Date: Jan. 20, 2005

(54) Pancreatic cancer treatment

(76) Inventors: Jin Wei Wang, San Diego, CA (US); Huaiyu Ma, San Diego, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/850,936

(22) Filed: May 20, 2004

Related U.S. Application Data
(60) Provisional application No. 60/472,529, filed on May 21, 2003.

(51) Int. Cl. A61K 31/7072

(52) U.S. Cl. 514/50

(57) ABSTRACT

N⁴ derivatives of the known antitumor compound CNDAC are useful in treatment of pancreatic cancer, especially as an adjuvant treatment and especially over long term administration.

### CROSS-REFERENCE

This application claims the benefit of priority to U.S. Provisional Patent Application No. 60/472,529, filed on May 21, 2003, which is hereby incorporated by reference in its entirety.

### STATEMENT REGARDING FEDERALLY SPONSORED

This was supported in part by U.S. National Cancer Institute Grants P30 CA 23100-1881 and R43-89779, and the government may have certain rights to the invention.

### TECHNICAL FIELD

The invention relates to the field of cancer treatment. More specifically, the invention concerns treating pancreatic cancer using analogs of cytidine that are protected from activity of cytidine deaminase by acylation at N⁴.

### BACKGROUND ART

Pancreatic ductal adenocarcinoma is one of the most lethal of human malignancies, accounting for over 30,000 deaths yearly in the United States alone. Upon diagnosis, only 10% to 15% of these cancers are typically found to be resectable, due to the presence of locally advanced disease or distant metastases. Currently, the most common strategy in the treatment of advanced pancreatic cancer is treatment with gemcitabine, an intravenously administered 2′-deoxycytidine nucleoside analog that induces apoptosis of human pancreatic cancer cells and can inhibit tumor growth and progression. Despite maximal medical or surgical management, however, results of the treatment of patients with pancreatic ductal adenocarcinoma are dismal; as a group, patients with this disease have a median survival under 21 months. Clearly, new, effective treatment strategies are required to combat this deadly disease.

Recently, a novel nucleoside analog, CS-682, has been described and has been shown to have potent antitumor activity in several subcutaneously-implanted solid tumor xenografts (Kaneko, M., et al., Proc. Amer. Assoc. Cancer Res. (1997) 38:679). CS-682 is an orally administered N⁴-palmitoyl derivative of (1-(2-C-cyano-2-deoxy-beta-D-arabino-pentofuranosyl)cytosine) (CNDAC), a 2′-deoxycytidine analog whose antitumor effect is thought to be due to both the ability to inhibit DNA polymerase and to its ability induce DNA self-strand breakage through incorporation of an active metabolite into the strands (Hanaoka, K., et al., Int. J. Cancer (1999) 82:226-236). Oral CS-682 has been shown to exhibit more potent cytotoxic activity than its parent compound against several tumor cell lines, including those of the stomach, lung, colon and breast.

Previous studies of the effects of CS-682, have primarily demonstrated efficacy of the drug in in vitro cell cultures and non-metastasizing subcutaneous xenograft models. Only one prior study has described the effects of CS-682 on liver metastasis (Wu, M., et al., Cancer Res. (2003) 63(10):2477-82). The effect of CS-682 on pancreatic cancer is not known.

### DISCLOSURE OF THE INVENTION

It has now been found that CS-682 and similarly protected analogs of CNDAC are effective in long-term control and inhibition of pancreatic cancer. This is particularly important as an adjuvant treatment—i.e., as a long-term chronic treatment to supplement primary removal of the tumor by surgery, chemotherapy or radiation therapy.

Thus, in one aspect, the invention is directed to a method to treat pancreatic cancer in a subject, which method comprises administering to a subject in need of such treatment a N⁴ substituted derivative of 1-(2-C-cyano-2-deoxy-beta-D-arabino-pentofuranosyl)cytosine (CNDAC) in an amount effective to inhibit or prevent the proliferation of said cancer.

In other aspects, the invention is directed to combining this treatment with administration of additional therapeutic agents, and in administering the treatment when the subject has been previously treated, for example, by surgery, to remove the primary tumor. In still another aspect, the invention relates to pharmaceutical compositions of these cytidine analogs in unit dosage amounts effective for adjuvant treatment of pancreatic tumors.

Another aspect of the disclosed invention is directed to a pharmaceutical composition designed for the adjuvant treatment of pancreatic cancer which comprises a unit dosage amount of N⁴ substituted derivative of CNDAC in admixture with at least one pharmaceutically acceptable excipient.

In another aspect, the disclosed invention is directed to a method to inducing DNA-self-strand breakage in a pancreatic cancer cell, comprising administering a pharmaceutical composition comprising a N⁴ substituted derivative of 1-(2-C-cyano-2-deoxy-β-D-arabino-pentofuranosyl)cytosine (CNDAC), in an amount effective to inhibit proliferation of one or more pancreatic cancer cells, to a subject in need thereof, whereby DNA-self-strand breakage in the pancreatic cancer cell is induced.

Another aspect of the invention is directed to a method of inhibiting pancreatic cancer metastasis, comprising administering a pharmaceutical composition comprising a N⁴ substituted derivative of 1-(2-C-cyano-2-deoxy-β-D-arabino-pentofuranosyl)cytosine (CNDAC), in an amount effective to inhibit metastasis of one or more pancreatic cancer cells, to a subject in need thereof.

### BRIEF DESCRIPTION OF THE DRAWINGS

FIGS. 1A and 1B are graphs showing that administration of significant amounts of CS-682 daily have little effect on body weight and thus are not toxic.

FIG. 2 shows survival times of mice with MIA-PaCa pancreatic cancer as prolonged by administering CS-682.

FIGS. 3A and 3B show the results of administering CS-682 on tumor growth. FIG. 3A shows photographs of tumors labeled with red fluorescent protein (RFP) as a function of time. As shown in FIG. 3A, at day 16, although control mice showed massive enlargement of the tumor, such enlargement did not occur in mice administered CS-682 until day 33. FIG. 3B is a graph of quantitative measure of tumor area as a function of time.

FIGS. 4A-4C shows the results of autopsy of control mice (4A) mice administered 40 mg/kg daily of CS-682 (4B), and mice administered 60 mg/kg of CS-682 daily (4C).

FIG. 5 shows the effect of CS-682 administration on metastases of the primary tumor.

FIG. 6 shows the effect of administering CS-682 on primary tumor weight.

### MODES OF CARRYING OUT THE INVENTION

According to the method of the invention, derivatives of cytidine analogs, in particular derivatives of CNDAC, that are protected at the N⁴ position from deamination are useful in non-toxic, sustainable treatment for managing pancreatic cancer, especially as an adjuvant to surgical or other removal of the primary tumor.

The compounds of the invention are derivatives of the cytidine analog CNDAC. These derivatives are protected at the N⁴ nitrogen of the cytosine moiety by acylation or other suitable protecting group, such as an alkyl group or alkenyl or alkynyl group. Polyunsaturated alkenyl and alkynyl groups may also be used. Preferably, however, the N⁴ nitrogen is protected by acylation, preferably by a long-chain fatty acid. Thus, suitable protected groups include alkyl (1-20C); alkenyl (2-20C); alkynyl (2-20C); acyl and unsaturated acyl (1-24C). These groups may further be substituted with physiologically compatible substituents such as halo, preferably fluoro, amino, alkylamino, hydroxy, alkoxy, and any other physiologically compatible substituent that does not impair the protective effect of the N⁴ substituent or interfere with the antitumor effect of the analog. Preferred substituents are residues of natively occurring fatty acids, (including unsaturated fatty acids) such as myristic, stearic, palmitic, and oleic acids. Particularly preferred is the compound CS-682 which is already recognized as an antitumor agent; in this derivative, the substituent is the acyl group derived from palmitic acid. Thus, preferred compounds of the invention include but are not limited to 1-(2-C-cyano-2-deoxy-β-D-arabino-pentofuranosyl)-N⁴-myristoylcytosine; 1-(2-C-cyano-2-deoxy-β-D-arabino-pentofuranosyl)-N⁴-stearoylcytosine; 1-(2-C-cyano-2-deoxy-β-D-arabino-pentofuranosyl)-N⁴-palmitoylcytosine and 1-(2-C-cyano-2-deoxy-β-D-arabino-pentofuranosyl)-N⁴-oleoylcytosine.

Methods to synthesize these compounds are well known to those of ordinary skill in the art. For example, see U.S. Pat. No. 5,691,319, which is hereby incorporated by reference in its entirety.

The ability to administer these compounds over a long period of time, due to their lack of toxicity, permits chronic treatment which keeps the proliferation of pancreatic tumor cells and their metastases at bay. Further, these derivatives are administrable orally which facilitates their use on a long-term basis. Thus, the compounds of the invention can be administered over periods of days, weeks, typically 1-2 weeks, months, typically 1, 2, 3, 4, 5, 6, 7, 8, 9, 10, 11, 12, 13, 14, 15, 16, 17, 18, 19, 20, 21, 22, 23 or more months to 2 or more years. Administration can be conducted on a daily basis or by any other protocol that is repetitive over the long term to effect the desired treatment.

The term “treatment” refers to affecting a positive result in a subject known to harbor pancreatic tumor cells. The positive effect may be tumor regression, inhibition of tumor growth, prevention or inhibition of metastasis formation, prolonged survival time, enhanced quality of life, or any other positive outcome of administering the pharmaceuticals of the invention.

“Adjuvant” treatment refers to treatment where the primary tumor has been removed or inhibited by chemotherapy, radiation therapy and/or surgery. Thus, the treatment according to the method of the invention when it is “adjuvant” treatment is conducted in concert with additional antitumor measures.

A number of chemotherapeutic agents are available for treating pancreatic cancer. Examples of the agents include but are not limited to gemcitabine, irinotecan, paclitaxel, flavopiridol, doxorubicin, idarubicin, vincristine, exatecan, and the like. These and other agents can be used in combination with the compounds of the invention to treat pancreatic cancer.

Radiotherapy is also frequently used to treat pancreatic cancer. The compounds of the present invention can be used as an adjunct to radiotherapy to increase the efficacy of both treatment protocols.

The dosage levels of the derivative compounds described herein depend on the choice of derivative itself, the severity of the subject's condition, the mode of administration, the overall health of the subject, and the judgment of the attending practitioner. Dosages in the range of 0.1-500 mg/kg per day, preferably 1-200 mg/kg per day are contemplated, although dosages outside this range may be indicated in any particular instance. While any route of administration may be employed, including delivery by injection, transmucosal routes of administration, transdermal routes of administration, including skin patches, suppositories, nasal sprays and the like, oral administration is preferred for its convenience and acceptability to the subject. Oral administration employs formulations that are suitable for such ingestion such as capsules, tablets, syrups, powders, and flavored compositions as is generally understood in the art. Suitable formulations for the type of molecule represented by the compounds of the invention are found in Remington's Pharmaceutical Sciences, latest edition, Mack Publishing Co., Easton, Pa., incorporated herein by reference.

Because of their low toxicity, the compounds of the invention may be administered over long periods of time on a daily basis. In particular, protocols which require daily administration or administration 1-5 times, preferably 1-2 times, more preferably 1 time daily are favored. In one suitable protocol, for example, the compound is administered 1-2 times daily in chewable flavored tablets each containing an amount of compound that is 20 mg/kg based on the weight of the subject. Thus, a single tablet designed for a 70 kg human would contain approximately 1.4 g of active ingredient. Lower dosages administered more frequently, for example, before and after meals represent an additional preferred protocol.

Treatment may be maintained for as long as necessary to prevent or inhibit the recurrence or metastasis of the tumor.

The following example is offered to illustrate but not to limit the invention.

### EXAMPLE 1

### Effect of CS-682 in a Mouse Model of Pancreatic Cancer

The model employed pancreatic tumors derived from the MIA-PaCa-2 pancreatic cancer cell line.

A. Preparation of the Model

The MIA-PaCa-2 pancreatic cancer cell line was obtained from the American Type Culture Collection (Rockville, Md.), maintained in DMEM media supplemented with 10% heat-inactivated fetal bovine serum and 1% penicillin and streptomycin (Gibco-BRL, Life Technologies, Inc., Grand Island, N.Y.) and cultured at 37° C. in a 5% CO₂ incubator.

The pDsRed-2 vector (Clontech Laboratories Inc., Palo Alto, Calif.) was used to engineer MIA-PaCa-2 clones stably expressing RFP. This vector expresses RFP and the neomycin resistance gene on the same bicistronic message. pDsRed-2 was produced in PT67 packaging cells. RFP transduction was initiated by incubating 20% confluent MIA-PaCa-2 cells with retroviral supernatants of the packaging cells and DMEM for 24 hours. Fresh medium was replenished at this time and cells were allowed to grow in the absence of retrovirus for 12 hours. This procedure was repeated until high levels of RFP expression, as determined using fluorescence microscopy, were achieved. Cells were then harvested by trypsin/EDTA and subcultured into selective medium that contained 200 μg/ml G418. The level of G418 was increased to 2000 μg/ml stepwise. Clones expressing high levels of RFP were isolated with cloning cylinders as needed, and were amplified and transferred using conventional culture methods. High RFP-expression clones were isolated in the absence of G418 for 10 passages to select for stable expression of RFP in vivo.

Male nude mice (NCr-nu) between 4-6 weeks of age were maintained in a barrier facility on HEPA-filtered racks. The animals were fed with autoclaved laboratory rodent diet (Teckland LM-485; Western Research Products, Orange, Calif.). Animal experiments were performed in accordance with the Guidelines for the Care and Use of Laboratory Animals (NIH Publication Number 85-23) under assurance number A3873-01.

Red-fluorescent human pancreatic cancer xenografts were established in nude mice by surgical orthotopic implantation (SOI). Briefly, MIA-PaCa-2-RFP tumors in the exponential growth phase, grown subcutaneously in nude mice, were resected aseptically. Necrotic tissues were cut away, and the remaining healthy tumor tissues were cut with scissors and minced into 1 mm³ pieces in RPMI 1640 medium. Mice were then anesthetized and their abdomens were sterilized with alcohol. An incision was then created through the left upper abdominal pararectal line and peritoneum. The pancreas was carefully exposed and two tumor pieces were transplanted onto the middle of the gland using a single 8-0 surgical suture (Davis-Geck, Inc., Manati, Puerto-Rico). The pancreas was then returned into the peritoneal cavity, and the abdominal wall and the skin were closed in two layers using 6-0 surgical sutures. All procedures were performed with a 7× microscope (Olympus) or standard surgical loupes.

B. Drug Dose, Route and Schedule

CS-682 (1-(2-C-cyano-2-deoxy-β-D-arabino-pentofuranosyl)-N⁴-palmitoylcytosine, Sankyo Pharmaceuticals, Tokyo) was administered by oral gavage. Prior to the first treatment, mice were randomized into eight groups of 10 mice each for treatment purposes.

Group 1 served as the negative control and did not receive treatment.

Groups 2, 3 and 4 received 40, 60 and 80 mg/kg/dose CS-682, respectively, each scheduled treatment day.

Group 4, 5, 6 and 7 received 20, 30, 40 and 50 mg/kg/dose CS-682 twice each treatment day, respectively.

Dosing was initiated five days after surgical orthotopic implantation, and was performed five days each week until death.

C. Evaluation Methods

External, in vivo whole body imaging, for at least once a week, mice were weighed and underwent external, in vivo imaging. This was performed in a fluorescent light box illuminated by fiberoptic lighting at 470 nm (Lightools Research, Encinitas, Calif.). Emitted fluorescence was collected through a long-pass filter GG475 (Chroma Technology, Battleboro, Vt.) on a Hamamatsu C5810 3-chip cooled color CCD camera (Hamamatsu Photonics Systems, Bridgewater, N.J.). High resolution images of 1024×724 pixels were captured directly on an IBM PC or continuously through video output on a high resolution Sony VCR model SLV-R1000 (Sony Corp., Tokyo, Japan). Images were processed for contrast and brightness and analyzed with the use of Image Pro Plus 3.1 software (Media Cybernetics, Silver Spring, Md.). Real-time determination of tumor burden was performed by quantifying fluorescent surface area, as described previously (Bouvet, M., et al., Cancer Res. (2002) 62:1534-1540).

For direct imaging and RFP fluorescence microscopy, mice were sacrificed and explored when they appeared pre-morbid. Following euthanasia, each mouse underwent laparotomy and sternotomy. Excitation of RFP in the light box, described above, facilitated identification of primary and metastatic disease by fluorescence visualization. After performing full-body, open images, the solid organs were removed and their surfaces were thoroughly examined for any evidence of metastases. Organs were then frozen and sliced into cross-sectional samples approximately 2 mm in width and visualized through a Leica fluorescence stereo microscope model LZ12 (Leica Microsystems, Inc., Bannockburn, Ill.) equipped with a mercury 50-W lamp power supply. Selective excitation of RFP was produced through a D425/60 band-pass filter and 470 DCXR dichroic mirror. Emitted fluorescence was collected by the Hamamatsu camera system described above.

D. Statistical Analysis

Differences among treatment groups were assessed using ANOVA and Student's t test using Statistica (Statsoft, Inc., Tulsa, Okla.). Kaplan-Meier analysis with a log rank test was used to determine survival and differences between treatment groups. A p≦0.05 was considered to be statistically significant.

Using the above techniques, the following results were obtained:

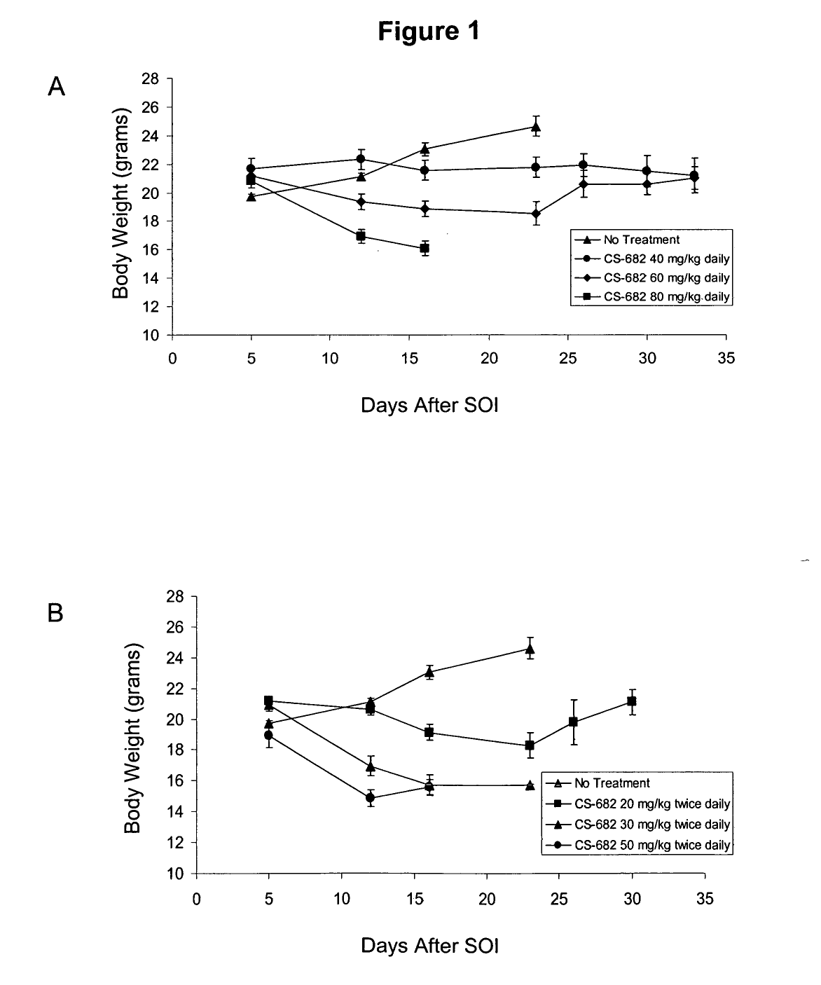

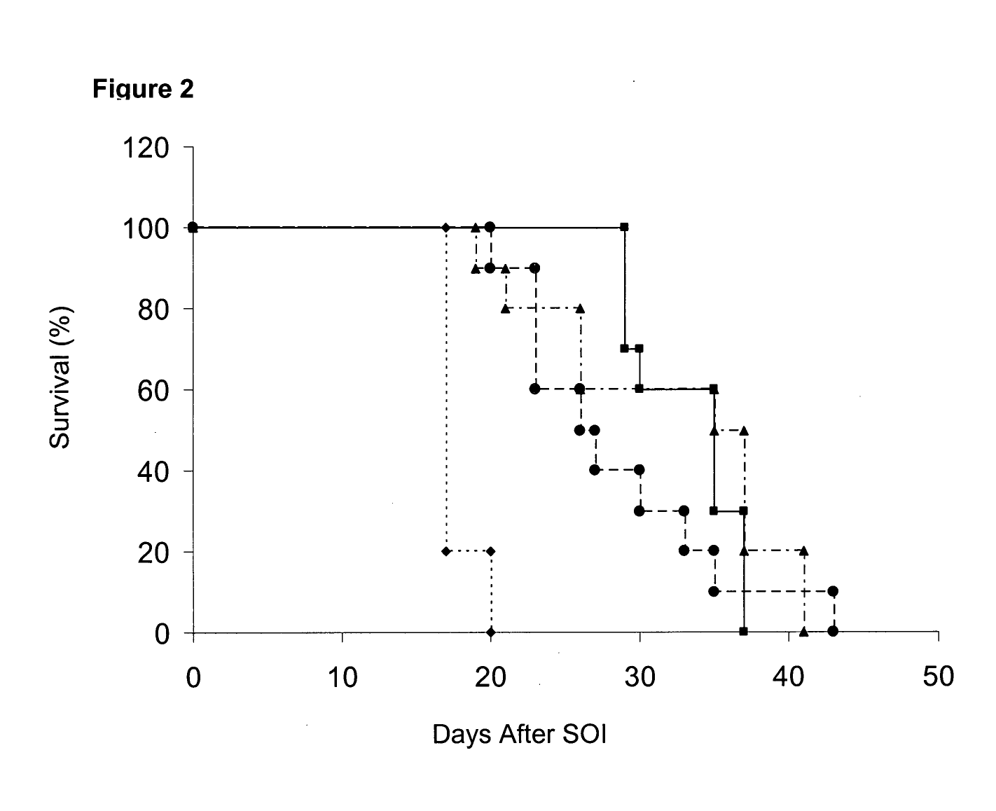

Body Weight Loss and Toxicity: At a CS-682 dose of 40 mg/kg once daily, no decrease in body weight was noted as shown in FIGS. 1A and 1B. FIG. 1A, shows the results using 40 mg/kg (closed circles), 60 mg/kg (diamonds), or 80 mg/kg (squares). FIG. 1B shows results with 20 mg/kg (squares), 30 mg/kg (triangles), and 50 mg/kg (circles), each administered twice daily. Diminution in body weight appears to occur at 60 mg/kg per day, but below this dose, the toxicity is insignificant. At higher doses, body weight declined to less than 80% of baseline. At these doses, toxicity-related death was observed in a significant number of mice. Notably, once-a-day dosing at 40 mg/kg and 60 mg/kg was associated with less weight loss and toxicity than was delivering the same dose divided twice daily.

Survival: Median survival of untreated mice with MIA-PaCa-2-RFP pancreatic cancer was 17 days. Oral administration of CS-682 significantly prolonged survival at several doses. These results are show in FIG. 2. Median survival was increased from 17 days in the control group to 27 days (p=0.003), 35 days (p=0.0008) and 36 days (p=0.002) at CS-682 dosages of 20 mg/kg twice daily, 40 mg/kg daily and 60 mg/kg daily, respectively. ♦=control, ●=CS-682 20 mg/kg twice daily, ▴=CS-682 60 mg/kg daily, ▪=CS-682 40 mg/kg daily. The significant increase in survival occurred despite the fact that several mice in the 60 mg/kg per day and 20 mg/kg twice-per-day treatment groups appeared to suffer toxicity-related death, since their primary and metastatic burden determined at autopsy were insufficient to explain their mortality. Prolongation of survival was not significantly different between these three treatment groups (p=0.19). At higher doses, survival was not enhanced by CS-682 administration, with a median survival of 18 days at a dose of 50 mg/kg twice daily and 19 days at doses of 30 mg/kg and 40 mg/kg twice daily and 80 mg/kg daily.

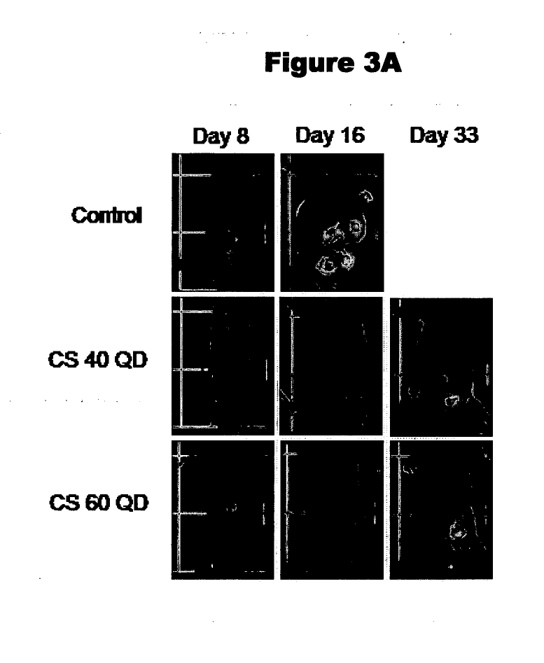

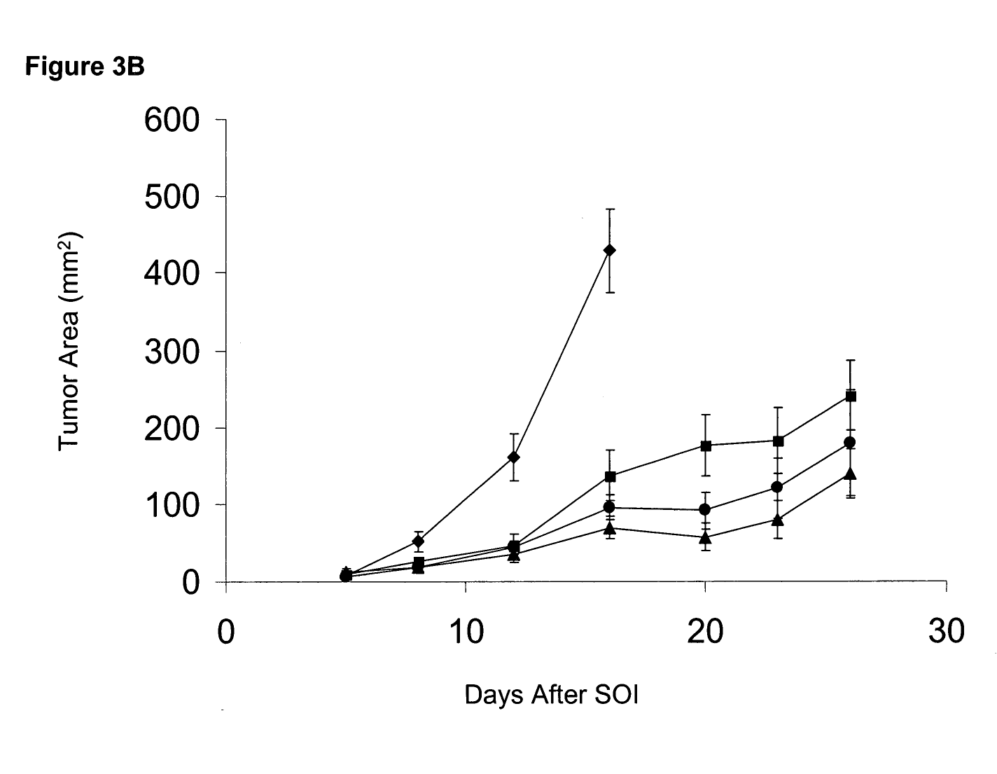

Tumor Growth and Metastasis: Tumor RFP autofluorescence enabled real-time, sequential whole-body imaging and quantification of tumor burden. In control mice, significant primary tumor growth and metastatic spread was visible within the first two weeks after surgical orthotopic implantation of tumor (FIG. 3A). On day 16 after SOI, each of these mice was identified to have disseminated metastatic disease, visualized externally by RFP fluorescence, in all four quadrants of the abdominal cavity. Additionally, the development of malignant ascites was found in 100% of control animals within the first 16 days after implantation.

In contrast, mice treated with CS-682 did not demonstrate significant tumor dissemination until the third and fourth week after implantation (FIG. 3A). Panels depict a representative mouse from each of three treatment groups on days 8, 16, and 33 after tumor implantation. Tumor dissemination in all four abdominal quadrants was visible within the first two weeks after implantation in the control group. In mice treated with CS-682 at 40 mg/kg and 60 mg/kg daily, widespread tumor metastasis was not visible until the third and fourth week post-implantation. By day 16, when control animals were found to have massive intra-abdominal dissemination of tumor, 90% of mice treated with 40 mg/kg daily CS682 were found to have locally confined disease. Accumulation of ascites was also less frequent in treated animals, with 50% and 10% of mice at treatment doses of 40 mg/kg and 60 mg/kg daily, respectively, having evident intra-abdominal fluid on examination.

Quantification of RFP autofluorescence facilitated real-time comparison of each treatment dose (FIG. 3B), demonstrating the ability of CS-682 to inhibit pancreatic cancer growth at dosages of 20 mg/kg twice daily, 40 mg/kg daily and 60 mg/kg daily (p<0.05 at each time point). Values represent the mean area of external RFP autofluorescence ±S.E. for live intact animals in each treatment group (p<0.05 for each time point). ♦=control, ●=CS-682 20 mg/kg twice daily, ▴=CS-682 60 mg/kg daily, ▪=CS-682 40 mg/kg daily.

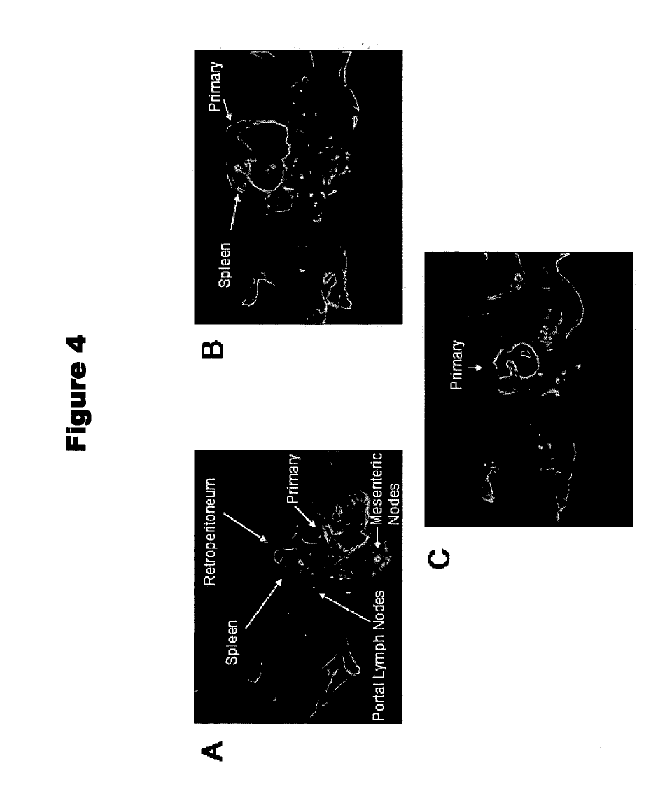

The animals were autopsied at death with the results shown in FIGS. 4A-4C. A, Control. B, CS-682 40 mg/kg daily. C, CS-682 60 mg/kg daily. Extensive primary tumor growth, as well as metastases to the diaphragm, peritoneum, liver, and mesenteric and portal lymph nodes were evident in almost all mice in the control group. Distant metastases were less frequent in mice treated with CS-682.

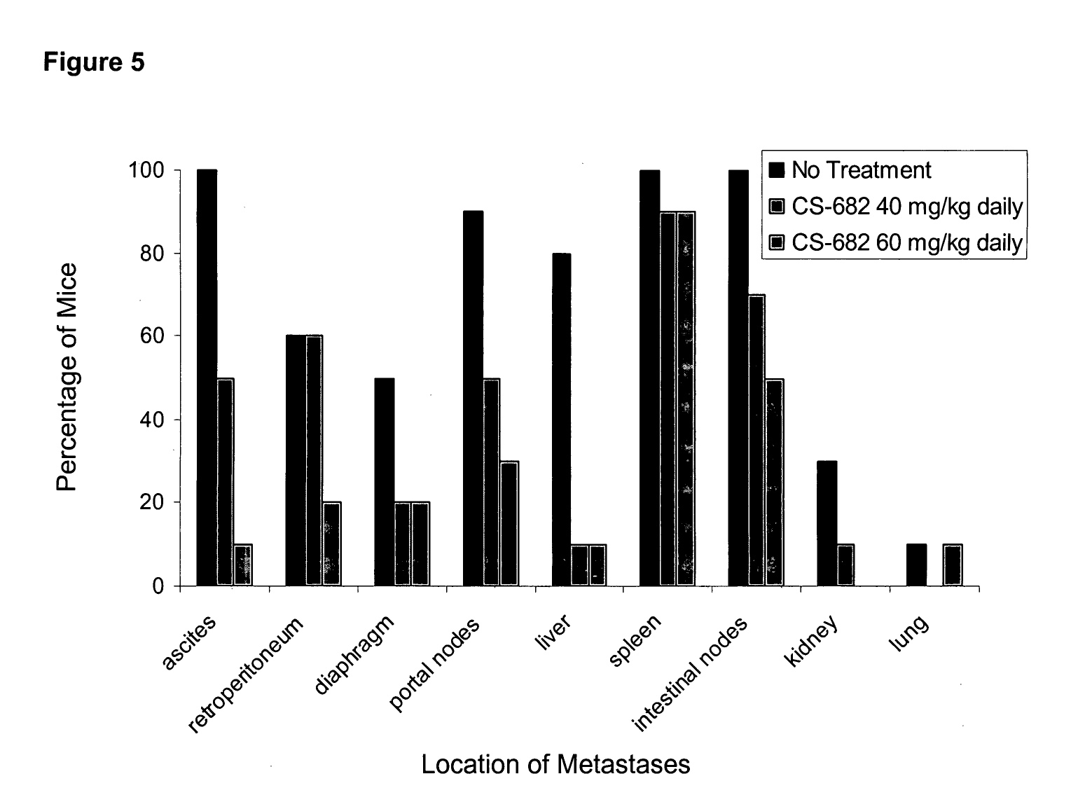

In untreated animals at the time of autopsy, metastases were found in the spleen (100%), intestinal nodes (100%), portal nodes (90%), liver (80%), retroperitoneum (60%), diaphragm (50%), kidney (30%) and lung (10%) (FIG. 5). In contrast, treatment with CS-682 at 40 mg/kg daily significantly inhibited the development of metastases in the diaphragm, portal nodes, liver, intestinal nodes and kidney. Dosages above 40 mg/kg daily further decreased the number of metastases found at autopsy, but with increased toxicity.

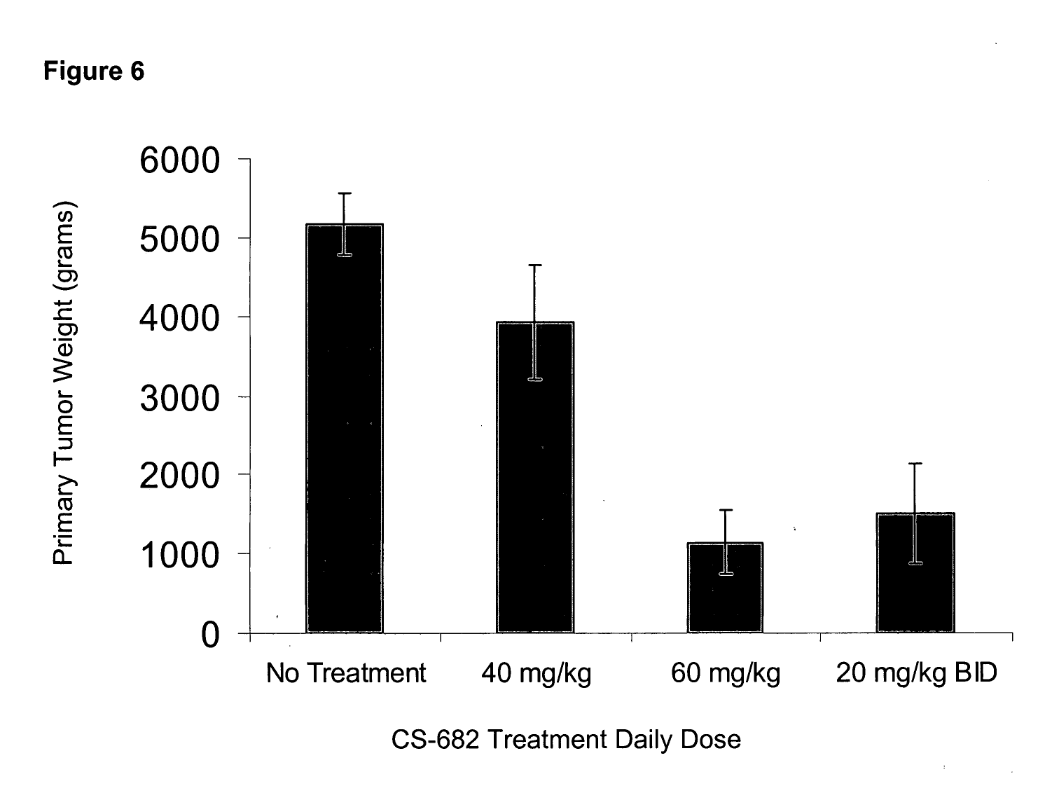

Although CS-682 did appear to have a growth-suppressive effect on the primary pancreatic tumor, this effect was not significant at 40 mg/kg daily (FIG. 6). At the time of autopsy, primary tumors in control mice had an average weight of 5.163 g, which was statistically similar to the primary weight of those mice treated with CS-682 at a dose of 40 mg/kg (3.935 g, p=0.16). Treatment with higher, toxic doses of CS-682 did appear to have a significant effect on primary tumor growth (60 mg/kg daily p=0.0004, 20 mg/kg twice daily p=0.003), but these doses appeared to lose tumor selectivity and were associated with significant toxicity.

### EXAMPLE 2

### Effect on Survival Efficacy of CS-682 Adjuvant Therapy in a Mouse Model of Metastatic Pancreatic Cancer

The efficacy of oral CS-682 in the adjuvant treatment of metastatic pancreatic cancer was studied. Administration of CS-682 as an adjuvant to surgical resection was shown to prolong survival compared to animals receiving no treatment, chemotherapy alone, or surgical resection alone. The study discussed below used a highly aggressive clone of the human pancreatic cancer cell line MIA-PaCa-2 pancreatic cancer cell line that was engineered, as discussed above, to selectively express high levels of the Discosoma red fluorescent protein. This brightly fluorescent model facilitated the noninvasive quantification of tumor burden throughout the course of treatment.

Experimental

A. Preparation of the Model

The MIA-PaCa-2 pancreatic cancer cell line and the animals used in the study were prepared as discussed in Example 1, with the following exceptions. Following orthotopic implantation of MIA-PaCa-2-RFP tumors, each mouse in a treatment group requiring surgical resection of the primary pancreatic tumor was anesthetized and prepared for surgery. The peritoneum was subsequently reopened through the original incision and an examination of adjacent structures was performed to ensure that macroscopic disease was localized to the pancreas. All grossly visible tumor was removed using sharp dissection. Hemostasis was achieved using 6-0 sutures. The abdomen was then closed in two layers.

Seven days after SOI, mice were randomized into eight groups of 10 mice each, depending upon whether they were to be treated by surgical resection, chemotherapy or both. Mice in groups 1-4 were not treated surgically. Mice in group 1 did not receive chemotherapy and thus served as negative controls. Mice in groups 2-4 were treated with primary CS-682 at doses of 40, 50 Or 60 mg/kg each treatment day, respectively, according to the treatment schedules outlined below.

Mice in groups 5-8 underwent surgical resection of their primary tumors 7 days after orthotopic implantation by a single blinded surgeon. Mice in group 5 received no additional chemotherapy; mice in groups 6-8 received adjuvant CS-682 at doses of 40, 50 or 60 mg/kg each treatment day, respectively.

CS-682 was administered by oral gavage. Treatment with primary or adjuvant CS-682 was initiated 9 days after orthotopic tumor implantation (2 days after surgical resection when applicable) and was to be administered five times each treatment week for a total of 5 weeks or until death. As detailed below, mice in groups receiving 60 mg/kg did not tolerate chronic treatment and required a modification of the treatment schedule. In these groups, CS-682 was administered 9 times in weeks 1 and 2 after SOI and then 10 times in weeks 4 and 5, with a treatment hiatus during week 3.

B. External in vivo Whole-Body Imaging

Twice/week, mice were weighed and underwent external in vivo imaging. This was performed in a fluorescent light box illuminated by fiberoptic lighting at 470 nm (Lightools Research, Encinitas Calif.). Emitted fluorescence was collected Emitted fluorescence was collected through a long-pass filter GG475 (Chroma Technology, Battleboro, Vt.) on a Hamamatsu C5810 3-chip cooled color charge-coupled device camera (Hamamatsu Photonics Systems, Bridgewater, N.J.). High-resolution images of 1024×724 pixels were captured directly on an IBM PC or continuously through video output on a high-resolution Sony VCR model SLV-R1000 (Sony Corp., Tokyo, Japan). Images were processed for contrast and brightness and analyzed with the use of Image Pro Plus 3.1 software (Media Cybernetics, Silver Spring, Md.). Real-time determination of tumor burden was performed by quantifying fluorescent surface area.

C. Direct Open Imaging and RFP Fluorescence Microscopy

Mice were sacrificed and explored when they appeared premorbid. After euthanasia, each mouse underwent laparotomy and sternotomy. Excitation of RFP in the light box, described above, facilitated identification of primary and metastatic disease by fluorescence visualization. After performing full-body open images, the solid organs were removed, and their surfaces were thoroughly examined for any evidence of metastases. Fluorescence microscopy was accomplished using a Leica fluorescence stereo microscope model LZ12 (Leica Microsystems, Inc., Bannockburn, Ill.) equipped with a mercury 50-W lamp power supply. Selective excitation of RFP was produced through a D425/60 band-pass filter and 470 DCXR dichroic mirror. Emitted fluorescence was collected by the Hamamatsu camera system described above.

D. Histological Analysis.

Representative primary tumors and metastases were removed at the time of autopsy, fixed in 10% formalin, embedded in paraffin, and sectioned. Samples were subsequently processed with standard H&E staining for Brightfield microscopic examination.

E. Statistical Analysis.

Differences among treatment groups were assessed using ANOVA and Student's t test using STATISTICA (Statsoft, Inc., Tulsa, Okla.). Kaplan-Meier analysis with a log-rank test was used to determine survival and differences between treatment groups. P 0.05 was considered to be statistically significant.

Results

A. Morphological and Growth Characteristics of MIA-PaCa-2-RFP in vitro.

RFP-expressing MIA-PaCa-2 cells appeared morphologically identical to their parent MIA-PaCa-2 cell line under light microscopy. The growth rates of MIA-PaCa-2 and MIA-PaCa-2-RFP cells was previously demonstrated to be statistically equivalent. Primary and metastatic MIA-PaCa-2-RFP pancreatic tumors exhibited features of poorly differentiated pancreatic ductal adenocarcinoma on H&E staining.

B. Analysis of Toxicity.

The body weight and general appearance of each mouse were monitored and recorded twice weekly as evidence of systemic toxicity. The weights of mice that did not receive CS-682 either remained constant until death or rose gradually due to the accumulation of intra-abdominal ascites. Wasting of body fat, most pronounced in the interscapular area of the back, was a common late finding that occurred in conjunction with disseminated disease.

At a dose of 40 mg/kg, treatment with CS-682 was not associated with a significant loss in body weight. As in control groups, interscapular wasting of body fat was a late finding and was not observed in the absence of disseminated disease. Death in all mice, even those receiving long-term treatment, clearly occurred from disseminated pancreatic cancer, not drug toxicity.

In groups treated with 50 mg/kg CS-682, the effects of chronic drug administration were not sufficient to require a modification of the original treatment protocol. Nonetheless, a moderate decrease in body fat with interscapular wasting was frequently noted in the absence of disseminated pancreatic disease after 2 weeks of continuous CS-682 treatment, indicating a cumulative effect of drug administration over time. This effect was not severe and did not lead to a significant loss of body weight. Even so, one mouse in each of the adjuvant and primary groups appeared to die from chronic drug toxicity, both after 2 complete weeks of chemotherapy.

Although no acute drug toxicity was observed in mice treated with 60 mg/kg CS-682, cumulative adverse effects were noted over the first 2 weeks of treatment in all mice, requiring termination of drug administration after the first nine doses. By this time, inter-scapular fat wasting was noted to be severe in all animals, leading to a 15% loss in body weight by day 20. At this point, administration of CS-682 was aborted until week 4, allowing all animals to recover with concurrent gains in both body fat and weight, after which, treatment resumed for 2 more weeks. Using this strategy, only 1 animal was lost to toxicity on day 41. Death in all other animals did not occur in the absence of disseminated pancreatic disease.

C. In vivo Characteristics of MIA-PaCa-2-RFP Tumor Growth.

Real-time, fluorescence whole-body optical imaging revealed a progressive increase in locoregional and metastatic growth in all un-treated animals after SOI of human MIA-PaCa-2-RFP pancreatic tumor fragments. Fluorescent primary tumor was visible through the skin as early as 5 days after implantation and was visible in 70% of animals by day 10 and 100% of animals by day 14. The development of distant solid tumor metastases and intra-abdominal ascites were both early findings, identified in 60 and 100% of animals, respectively, by day 16. Noninvasive quantitative measurements of externally visible fluorescent area enabled the construction of in vivo tumor growth curves, which demonstrated a remarkably linear tumor growth rate in the untreated animals that led to death from disease in all mice by 30 days and a median survival of 26 days. Upon autopsy, metastases were confirmed in multiple sites, including the diaphragm, intestinal and portal lymphatics, retroperitoneum, kidney, and liver.

D. Surgical Resection.

Early surgical resection of primary disease led to a marginal yet significant increase in survival (median survival, 28 days, P 0.03). In all cases, resection was performed before accumulation of any distant metastatic deposits and entailed removal of all gross pancreatic disease, with a concurrent, significant reduction in fluorescent tumor area visible externally by day 10 (P 0.009). Nonetheless, the benefits of surgery were clearly transient. Recur-rent tumor burden increased progressively after day 10, reaching preoperative levels by day 14. At this point, tumor enlargement and dissemination accelerated, with an average tumor growth rate similar to that seen in the no treatment group. As expected, surgical resection also postponed the development of ascites, with only 20% of animals exhibiting this clinical finding on day 16.

E. Primary CS-682 Treatment.

In concordance with our previous results (13), primary administration of CS-682 at each dose tested significantly enhanced the survival of mice with orthotopically implanted MIA-PaCa-2-RFP tumors (P 0.05 for each dose). The survival advantage conferred by CS-682 was similar using each dose tested (P 0.4). At doses of 50 and 60 mg/kg, a significant increase in overall survival was also achieved over surgical resection alone (P 0.045 and 0.03, respectively) by the primary administration of CS-682.

Real-time whole-body imaging of early tumor growth confirmed that the favorable effect of CS-682 chemotherapy on survival was caused by a significant reduction in the rate of tumor growth by this agent. Although mice treated primarily with CS-682 had more tumor than those treated surgically over the first 2-3 weeks after implantation, the growth-suppressive effects of CS-682 outlasted the transient effects of surgical tumor resection, with an intersection in the growth curves at 21 days.

F. Resection and Adjuvant CS-682 Treatment.

The largest increase in survival was achieved by the postoperative administration of CS-682 after surgical resection. At all doses tested, mice treated in this manner had a significant increase in survival over treatment controls (P 0.05). On the 50 and 60 mg/kg regimens, the enhancement in survival was also significant compared with resection alone (P 0.004 and 0.03, respectively). Enhancement of survival was most significant at a dose of 50 mg/kg. On this regimen, mice had a median survival of 48 days and 30% lived at least 60 days. Using fluorescence visualization, the growth-suppressive effects of this combination therapy was evident: on day 23, after 40% of untreated animals had already succumbed to disseminated disease, 70% of animals in the adjuvant 50 mg/kg group had, at most, local disease confined to one abdominal quadrant. In the 30% of mice that experienced particularly long-term survival (60 days) on this regimen, distant metastasis was not seen until 10 days after the completion of chemotherapy, at which time, the development of metastasis accelerated, and the mice ultimately succumbed to disseminated pancreatic disease. Construction of in vivo tumor growth curves demonstrated apparent synergism between surgical resection and the application of adjuvant CS-682 in the suppression of tumor growth. As expected, the development of ascites was also suppressed by adjuvant therapy, with only 20% of animals showing signs of fluid retention at the time of their death.

The examples disclosed are provided solely to exemplify the disclosed invention, which is to be limited by the language of the claims.

1. A method to treat pancreatic cancer comprising:
administering a pharmaceutical composition comprising a N⁴ substituted derivative of 1-(2-C-cyano-2-deoxy-β-D-arabino-pentofuranosyl)cytosine (CNDAC), in an amount effective to inhibit proliferation of one or more pancreatic cancer cells, to a subject in need thereof.

2. The method of claim 1, wherein the pharmaceutical composition administered is adapted for oral administration.

3. The method of claim 1, wherein the N⁴ substituted derivative of the pharmaceutical composition administered comprises a CNDAC coupled through the N⁴ position to a substituent that is optionally substituted alkyl (1-20C); alkenyl (2-20C); alkynyl (2-20C); acyl or unsaturated acyl (1-24C).

4. The method of claim 3, wherein said substituent is an acyl or unsaturated acyl.

5. The method of claim 4, wherein said acyl is the residue of palmitic, myristic, stearic or oleic acid.

6. The method of claim 5, wherein said derivatized CNDAC is CS-682 (1-(2-C-cyano-2-deoxy-β-D-arabino-pentofuranosyl)-N⁴-palmitoylcytosine).

7. The method of claim 1, wherein said subject has been treated with chemotherapy, radiation therapy, and/or surgery to resect said one or more pancreatic cancer cells.

8. The method of claim 1 which further comprises administering an antitumor agent.

9. A pharmaceutical composition designed for the adjuvant treatment of pancreatic cancer which comprises a unit dosage amount of N⁴ substituted derivative of CNDAC in admixture with at least one pharmaceutically acceptable excipient.

10. The composition of claim 9, wherein said derivative is CS-682.

11. The composition of claim 9 which is suitable for oral administration.

12. The composition of claim 9 which is suitable for oral administration.

13. A method to inducing DNA-self-strand breakage in a pancreatic cancer cell, comprising:
administering a pharmaceutical composition comprising a N⁴ substituted derivative of 1-(2-C-cyano-2-deoxy-β-D-arabino-pentofuranosyl)cytosine (CNDAC), in an amount effective to inhibit proliferation of one or more pancreatic cancer cells, to a subject in need thereof, whereby DNA-self-strand breakage in the pancreatic cancer cell is induced.

14. The method of claim 13, wherein the pharmaceutical composition administered is adapted for oral administration.

15. The method of claim 13, wherein the N⁴ substituted derivative of the pharmaceutical composition administered comprises a CNDAC coupled through the N⁴ position to a substituent that is optionally substituted alkyl (1-20C); alkenyl (2-20C); alkynyl (2-20C); acyl or unsaturated acyl (1-24C).

16. The method of claim 15, wherein said substituent is an acyl or unsaturated acyl.

17. The method of claim 16, wherein said acyl is the residue of palmitic, myristic, stearic or oleic acid.

18. The method of claim 17, wherein said derivatized CNDAC is CS-682 (1-(2-C-cyano-2-deoxy-β-D-arabino-pentofuranosyl)-N⁴-palmitoylcytosine).

19. The method of claim 18, wherein said subject has been treated with chemotherapy, radiation therapy, and/or surgery to resect said one or more pancreatic cancer cells.

20. The method of claim 19 which further comprises administering an antitumor agent.
